(19) United States
(12) Patent Application Publication
(10) Pub. No.: US 2017/0209577 A1
(43) Pub. Date: Jul. 27, 2017

(54) DIRECTED DELIVERY OF ACTIVE AGENTS USING QUANTUM DOTS

(71) Applicant: Vardhaan S. Ambati, San Jose, CA (US)

(72) Inventors: Vardhaan S. Ambati, San Jose, CA (US); Johan Sosa, San Jose, CA (US)

(21) Appl. No.: 15/004,714

(22) Filed: Jan. 22, 2016

(51) Int. Cl. A61K 41/00; A61N 5/06

(52) U.S. Cl. CPC A61K 41/0042; A61N 5/062

(57) ABSTRACT

This invention relates to the use of functionalized quantum dots to effect the targeted delivery of active agents to a diseased cell.

### FIELD

The field of this invention is the delivery of active agents to specific targets in a subject using quantum dots as active agent carriers.

### BACKGROUND

In the treatment of drug-susceptible diseases and disorders, one of the key concerns is drug delivery. While many factors are involved in drug delivery, such as absorption, distribution, metabolism, elimination, the so-called ADMET factors, the fundamental decision regarding drug delivery, which affects all other factors, is route of delivery. Drug delivery is generally divided into two broad categories, systemic and local. Systemic delivery generally implies administration through the circulatory system, either enteral (absorption through the gastrointestinal tract) or parenteral (injection, infusion or implantation). Systemic administration is often preferred due to non-invasiveness, convenience and patient acceptance. Peroral (by mouth), topical, nasal, buccal/sublingual and ocular administration are examples of systemic delivery. Systemic delivery, however, is generally not the most efficient and/or economical method of administration. That is, sufficient drug must be administered to assure that a pharmaceutically effective amount reaches the target taking into account loss of the drug due to metabolism, entrapment in non-target organs or premature excretion. Toxicity is also an issue since unintended targets may be deleteriously affected. Further, some pharmaceuticals, such as peptides and proteins, which tend to be particularly susceptible to enzymatic degradation are simply not amenable to systemic delivery.

An alternative to systemic delivery is localized delivery in which the active agent is delivered as close to the target site as possible. This approach tends to mitigate most of the disadvantages of systemic delivery but is generally relatively invasive and therefore not particularly liked by patients. Numerous approaches to localized delivery have been developed, for instance, use of a catheter that is advanced to a point close to the target and then the drug is released or simply injecting the drug directly to the target site using a syringe or micro-needle array.

A more recent addition to the drug administration repertoire is targeted drug delivery, which lessens the gap between systemic and local drug delivery and makes patient preferred systemic delivery more feasible. As the name implies, the goal of targeted dug delivery is to deliver an active agent solely to diseased tissue. Such result has been successfully achieved by, for example, attaching active agents to carrier nanoparticles, which also have ligands attached to them that are specific to receptors on the diseased target cells. Once the nanoparticle is docked on the target cell, the active agent must be released to have its therapeutic effect. This can be accomplished in a number of ways including chemical release, e.g., pH sensitivity of the bond between the nanoparticle and the active agent, enzymatic release and, more recently, photochemical release. Photochemical release involves formation of a photocleavable link between the active agent and the carrier. Then, once the carrier has reached the diseased tissue, the area is irradiated at a wavelength that causes the photocleavable bond to break, releasing the active agent. A problem associated with photochemical release, however, is getting the proper wavelength of light to the target site in sufficient intensity to effect release of the drug from the carrier. Generally speaking, ultraviolet (uv) light effectively cleaves a substantial range of photocleavable bonds. There are, however, photocleavable linkages that require wavelengths of light beyond uv, even into the visible spectrum, to achieve scission. Such wavelengths of light may, however, be difficult to deliver to a diseased tissue due to lack of penetrability through surrounding unaffected tissue and lack of sufficient intensity at the target site. What is needed is a technique that that increases the spectrum of light that can be used to effect cleavage of photolabile groups and simultaneously provide sufficient quantities of the desired wavelength at the target site to assure cleavage. The present invention provides such a composition and method.

### SUMMARY

Thus, in one aspect, the present invention is directed to a composition for targeted delivery of an active agent, comprising:
a biocompatible quantum dot having an outer surface;
a targeting entity bound to the outer surface of the quantum dot, which targeting entity is selected so as to bind to a cell-specific receptor of a target cell;
a linker having a first binding site, which binding site binds the linker to the outer surface of the quantum dot;
an active agent bound to a second binding site of the linker, the second binding site comprising a photocleavable chemical bond; wherein:
   the quantum dot is of a size such that its fluorescence wavelength is the scission wavelength of the photocleavable chemical bond.

In as aspect of this invention, the composition further comprises a cell-penetrating entity bound to the outer surface of the quantum dot.

In an aspect of this invention, the targeting entity is selected from the group consisting of an antibody, an aptamer, and an affibody.

In an aspect of this invention, the targeting entity is an aptamer.

In an aspect of this invention, the first binding site on the linker is selected from the group consisting of a thiol, a mercaptoacetyl, avidin or streptavidin when the surface of the quantum dot comprises biotin and biotin when the surface of the quantum dot comprises avidin or streptavidin.

In an aspect of this invention, the active agent is selected from the group consisting of a drug, an extra-cellular signaling agent and a non-self antigen.

In an aspect of this invention, the drug is a cytostatic or cytotoxic drug.

An aspect of this invention is a method of treatment of a cell-based disease or disorder, comprising:
providing a plurality of biocompatible quantum dots that fluoresce at a selected wavelength when irradiated;
binding a targeting entity to each of the quantum dots, wherein:
   the targeting entity has an affinity for cells displaying a unique cell surface receptor;
binding a linker to the surface of the quantum dot through a first binding site of the linker;
binding a therapeutic agent to the linker through a second binding site of the linker, wherein:
   the second binding site comprises a photocleavable chemical bond that will cleave when irradiated at the fluorescence wavelength of the quantum dots;
administering the plurality of quantum dots modified as above to a subject in need thereof;
irradiating the subject with a wavelength of light that is absorbed by the quantum dots and results in the quantum dots fluorescing at the selected wavelength, wherein
   the photocleavable bond is broken and the bioactive agent is released.

In an aspect of this invention, the disease or disorder is selected from the group consisting of cardiovascular disease, diabetes and cancer.

In an aspect of this invention, the disease or disorder comprises cancer.

In an aspect of this invention, the cancer comprises a malignant tumor.

### DETAILED DESCRIPTION

It is understood that use of the singular throughout this application including the claims includes the plural and vice versa unless expressly stated otherwise. That is, “a” and “the” are to be construed as referring to one or more of whatever the word modifies. Non-limiting examples are: “an agent,” which is understood to include one or more such agents, and “a targeting entity” refers to one or more targeting entity, unless it is expressly stated or is unambiguously obvious from the context that such is not intended.

As used herein, words of approximation such as, without limitation, “about,” “substantially,” “essentially” and “approximately” mean that the word or phrase modified by the term need not be exactly that which is written but may vary from that written description to some extent. The extent to which the description may vary will depend on how great a change can be instituted and still have one of ordinary skill in the art recognize the modified version as still having the properties, characteristics and capabilities of the modified word or phrase. In general, but subject to the preceding discussion, a numerical value herein that is modified by a word of approximation may vary from the stated value by ±15%.

As used herein, the use of “preferred,” “preferably,” or “more preferred,” and the like refer to preferences as they existed at the time of filing of this patent application.

As used herein, the term “composition” refers to a mixture of the quantum dots of this invention with other chemical components, such as diluents, carriers, excipients or any combination of these that may facilitate administration of the quantum dots to a subject.

As used herein, a “subject,” refers to any organism that may benefit from treatment of a condition by administering an active agent by means of a composition of this invention. A presently preferred subject is a mammal and, most preferably at present, a human being. A subject herein may also be referred to as a “patient” the terms being interchangeable.

As used herein, “biocompatible” refers a quantum dot that, in its intact form, and in the form or forms it assumes, if any, during or as the result of decomposition or degradation is/are not harmful to a living organism.

As used herein, when one entity such as, without limitation, a QD, an active agent or an cell-penetrating entity, is said to “bind to” or “be bound to” another entity, the term “bind” or “bound” includes both chemical bond formation and physical interaction, such as non-covalent affinity binding. Context will unambiguously reveal which type of binding is concerned with which pair of substances that are bound.

As used herein, the terms “drug,” “active agent” and “therapeutic agent” are used interchangeably.

As used herein, “active agent” refers to any substance that, when administered in a therapeutically effective amount to a patient suffering from a condition, has a therapeutic beneficial effect on the health and well-being of the patient. A therapeutic beneficial effect on the health and well-being of a patient includes, but it not limited to: (1) curing the condition; (2) slowing the progress of the condition; (3) causing the condition to retrogress; or, (4) alleviating one or more symptoms of the condition. As used herein, an active agent also includes any substance that when administered to a patient, known or suspected of being particularly susceptible to a condition, in a prophylactically effective amount, has a prophylactic beneficial effect on the health and well-being of the patient. A prophylactic beneficial effect on the health and well-being of a patient includes, but is not limited to: (1) preventing or delaying on-set of the condition in the first place; (2) maintaining a condition at a retrogressed level once such level has been achieved by a therapeutically effective amount of a substance, which may be the same as or different from the substance used in a prophylactically effective amount; or, (3) preventing or delaying recurrence of the condition after a course of treatment with a therapeutically effective amount of a substance, which may be the same as or different from the substance used in a prophylactically effective amount, has concluded.

An “active agent” herein may comprise any pharmaceutic agent that is known or suspected to have a beneficial affect with regard to a cell-borne disease or disorder. In particular, pharmaceuticals that are effective against malignant tumors are presently preferred. There pharmaceuticals may be cytostatic, that is drugs that inhibit cell growth and division, or cytotoxic, drugs that kill cells. For the purpose of this invention, an active agent may also include a signaling agent or an antigen.

An active agent may also comprise a photodynamic therapy (PDT) sensitizer. In this option, a relatively non-toxic, inactive dye is bound to the linker. When the quantum dot is irradiated at the appropriate wavelength, it fluoresces at its emission wavelength, which wavelength is selected so as to excite the photosensitizer. This excitation results in the generation of highly cytotoxic singlet oxygen species that attack various components of the cell in which the QD has localized by virtue of the targeting agent.

As used herein, an “extra-cellular signaling agent” refers to molecule that activates a specific receptor located on a cell surface or inside a cell, wherein when triggered by the signaling the receptor initiates a cascade of events within the cell that results in a response to the signaling agent. Extracellular signaling agents include, without limitation, hormones, neurotransmitters or paracrine/autocrine agents. Extra-cellular receptors include, without limitation, G protein-coupled, tyrosine and histine kinase and integrin receptors.

As used herein, a “non-self antigen” refers to an antigen that originates in the external environment and that causes the immune system to produce antibodies against it.

As used herein, “treating” refers to the administration of a therapeutically effective amount of an active agent to a subject known or suspected to be suffering from a condition with regard to which a composition of this invention comprising such active agent has been demonstrated, or is deemed likely, to be effective.

A “therapeutically effective amount” refers to that amount of an active agent that will have a beneficial effect, which may be curative or palliative, on the health and well-being of the patient known or suspected to be afflicted by a particular disease or disorder. For the purpose of this invention, the therapeutically effective amount may be administered as a single bolus or as intermittent bolus charges.

As used herein, a ‘quantum dot” or “QD” refers to a semiconductor nanocrystal having a core diameter from about 1 to about 10 nm in diameter. They are comprised of two types of atoms, one from the Group II elements and one from the Group VI elements (for example, Cd/S, Cd/Se, Zn/S, Zn/Se, Cd/Te), the Group III and Group V elements (for example, In/P, In/As) or IV-VI elements (for instance, Pb/Se, Pb/S). Cd-free QDs are of particular interest with regard to this invention due to the absence of Cd, a known heavy metal toxin. The most widely studied QDs, however, still comprise Cd, such as, for example, the Cd/Se core/ZnS shell QD. The Cd/Se QD is generally passivated, that is, protected from deleterious interaction with environmental factors such as air and water, with a monolayer surface coating of a material such as tri-n-octylphosphine oxide (TOPO). QDs exhibit a most unique feature that render them particularly useful in the compositions and methods of this invention. That is, QDs, exhibit extremely high brightness when excited. This is due to their unusually high quantum yields coupled with a very high extinction coefficient. Further, QDs are resistant to photobleaching such that their fluorescence can persist for extended periods of time compared to traditional small molecule fluorphores. In addition, the absorption wavelength and fluorescence wavelength of QDs is precisely tunable based on the diameter of the QD. That is, smaller diameter QDs absorb and emit high energy bluer light while larger diameter QDs absorb and emit in the lower energy, redder portion of the spectrum. Since the absorption spectrum is also controlled by the size of the QD, QDs can be excited by exposure to white light, such as that of a while light laser. The emission spectrum of QDs tends to be Gaussian and does not exhibit the “shoulder” commonly encountered with typical emission spectra of organic dyes.

QDs, such as TOPO passivated QDs, are highly hydrophobic so, to achieve water solubility QDs are often encapsulated in amphiphilic molecules, the hydrophobic segment of which interacts with the hydrophobic TOPO at the QD surface and the hydrophilic segment imparts water solubility to the QD.

Of particular interest with respect to this invention are amphiphilic molecules that carry reactive groups, such as, without limitation, hydroxyl, amino and carboxylic acids as elements of their hydrophilic segments. As used herein, molecules of this sort are termed “linkers” and generally comprise biocompatible molecules one segment of which can interact with the surface of a QD and another segment of which can interact with a targeting entity or an active agent, thus binding the targeting entity and the active agent to the QD. A linker may comprise covalent bonds or non-covalent affinity-bonding that conjugates a biomolecule active agent to a QD by, without limitation, the avidin/biotin, streptavidin/biotin or nickel nitrilotriacetic acid (Ni-TA)/histidine-tagged peptide interaction. When a linker is described as having a “first binding site” and a “second binding site” it is understood that the first binding site is located at or near the proximal end of the linker molecule and the second binding site is located at or near the distal end of the linker molecule.

As used herein, a “targeting entity” refers to a biomolecule that is a receptor-specific ligand for a surface receptor either entirely unique or markedly over-expressed by a particular target cell, the receptor being referred to at times herein as a “cell-specific receptor.” While over-expression does not insure complete isolation of a composition hereof to specific target cells, over time the composition will naturally be included to concentrate where the concentration of surface receptors is most prevalent. Targeting entities include, without limitation, antibodies, aptamers and affibodies. A non-limiting example of a targeting entity is transferrin, which has been used to target tumor cells that possess a transferrin-receptor-mediated endocytosis mechanisms on their outer membrane. Many other such cell-specific receptors such as HER-2 and G protein-coupled receptors (GPCR) and entities that target them are known to those skilled in the art as are ligands that have a specific affinity for such receptors and their use with the compositions and methods of this invention will be clear based on the disclosures herein.

As used herein, “targeted delivery” refers to a method of delivering an active agent hereof in a manner that increases the concentration of the active agent in a specific diseased region of the body relative to other non-afflicted regions. By reducing the concentration of the active agent in non-afflicted tissues, targeted delivery also helps ameliorate side-reactions of active agents.

As used herein, a “cell-penetrating entity” refers to a molecule that assists in the internalization of a bioactive cargo into a target cell. An example of such a molecule is a cell-penetrating peptide, many examples of which are disclosed in the literature and need no further elucidation herein.

An embodiment of this invention comprises a linker for binding a drug to a QD wherein the second binding site of the linker is bound to the active agent comprises a photocleavable chemical bond. Myriad photocleavable covalent groups are known such as, without limitation, o-nitrobenzyl alcohol derivatives, a-ketoester derivatives, benzoin esters, fluorenecarboxylate, cinnamyl esters and vinylsilanes. Of particular interest for the purposes of this invention, however, are photocleavable bonds that break upon irradiation with visible light, which, among other advantages, eliminates the biological effects of uv irradiation. Such a linkage is described in U.S. patent application Ser. No. 14/432,403, which is incorporated by reference herein, and those described in Hossion, et. al., Visible light controlled release of anti-cancer drug through double-activation of prodrug, ACS Medicinal Chemistry Letters, 2013, 4(1):124-127, which likewise is incorporated by reference herein.

1. A composition for targeted delivery of an active agent, comprising:
a biocompatible quantum dot having an outer surface;
a targeting entity bound to the outer surface of the quantum dot, which targeting entity is selected so as to bind to a cell-specific receptor of a target cell;
a linker having a first binding site, which binding site binds the linker to the outer surface of the quantum dot;
an active agent bound to a second binding site of the linker, the second binding site comprising a photocleavable chemical bond; wherein:
the quantum dot is of a size such that its fluorescence wavelength is the scission wavelength of the photocleavable chemical bond.

2. The composition of claim 1 further comprising a cell-penetrating entity bound to the outer surface of the quantum dot.

3. The composition of claim 1 wherein the targeting entity is selected from the group consisting of an antibody, an aptamer, and an affibody.

4. The composition of claim 3, wherein the targeting entity is an aptamer.

5. The composition of claim 1, wherein the first binding site on the linker is selected from the group consisting of a thiol, a mercaptoacetyl, avidin or streptavidin when the surface of the quantum dot comprises biotin and biotin when the surface of the quantum dot comprises avidin or streptavidin.

6. The composition of claim 1, wherein the active agent is selected from the group consisting of a drug, an extra-cellular signaling agent and a non-self antigen.

7. The composition of claim 6, wherein the drug is a cytostatic or cytotoxic drug.

8. A method of treatment of a cell-based disease or disorder, comprising:
providing the composition of of claim 1, wherein the composition fluoresces at a selected wavelength when irradiated;
binding a targeting entity to a quantum dot in the composition, wherein:
the targeting entity has an affinity for cells displaying a unique cell surface receptor;
binding a linker to the surface of the quantum dot through a first binding site of the linker;
binding a therapeutic agent to the linker through a second binding site of the linker, wherein:
the second binding site comprises a photocleavable chemical bond that will cleave when irradiated at the selected wavelength;
administering the composition to a subject in need thereof; and
irradiating the subject with a wavelength of light that is absorbed by the quantum dot and results in the quantum dot fluorescing at the selected wavelength, wherein
the photocleavable bond is broken and the bioactive agent is released.

9. The method of claim 8, wherein the disease or disorder is selected from the group consisting of cardiovascular disease, diabetes and cancer.

10. The method of claim 8 wherein the disease or disorder comprises cancer.

11. The method of claim 10, wherein the cancer comprises a malignant tumor.
